(19) United States
(12) Patent Application Publication
(10) Pub. No.: US 2003/0055394 A1
(43) Pub. Date: Mar. 20, 2003

(54) Diaper tab grips

(76) Inventor: Bernadette M. Gibbs, Statham, GA (US)

Correspondence Address: Christopher C. Campbell, Esq., Hunton & Williams, Suite 1200, 1900 K Street, NW, Washington, DC, 20006-1109, US

(21) Appl. No.: 09/953,950

(22) Filed: Sep. 18, 2001

(51) Int. Cl. A61F 13/15

(52) U.S. Cl. 604/389; 604/391

(57) ABSTRACT

The present invention provides an absorbent article having first and second waist regions, and a crotch region between the first and second waist regions. The article generally comprises a topsheet, a backsheet, and an absorbent core disposed between the topsheet and the backsheet. At least one fastening element each is attached to a pair of tabs that extend laterally away from the first waist region. The fastening elements attach to one or more target devices that are located in the second waist region to form a pant-like garment on a wearer. A pair of grips are attached to opposite lateral sides of the first waist region. Each grip is attached in an attachment area located inboard of at least one of the respective fastening elements.

### FIELD OF THE INVENTION

The present invention generally relates to absorbent garments. In particular, the invention relates to an absorbent garment having a unique gripping device associated with the fasteners.

### BACKGROUND OF THE INVENTION

Absorbent garments, such as diapers and adult incontinence products, often are constructed to have an hourglass or rectangular shape that is fitted between a wearer's legs, then wrapped around the wearer's waist, and then sealed on either side to form a pant-like structure on the wearer. Many of these garments have an elasticized waist that the caregiver stretches out while placing the garment on the wearer. Fasteners typically are located in the waist region, or on tabs extending therefrom, for attaching one half of the garment to the other half. The tabs also may be elasticized.

In order to stretch the elasticized waist region and/or tabs, the caregiver typically must grasp either lateral edge of the garment and pull outwards. Often, the only convenient places at which the caregiver can grasp the garment are the laterally distal edges of the fasteners. In doing so, the caregiver places the fasteners under a tensile load which may deform the tabs and the fasteners, thereby inhibiting the proper attachment of the fasteners. In addition, the caregiver may have to pinch the garment between his or her fingers, and the caregiver's fingers or thumbs may partially or completely cover the fasteners while the caregiver attempts to secure the fasteners. As the caregiver affixes some portions of the fasteners, other portions of the fasteners may form wrinkles that are not fully attached to the garment. Other portions of the fastener may be attached while the waist or tab elastic is in different states of stretch, thereby causing different loading in different parts of the fastener. Still other portions of the fastener may not be attached at all, and may even turn to face outward due to the tensile load in the elastics or other loads generated during use. In any of these cases, and others not mentioned herein, the fasteners may not attach the two halves of the garment to one another in an ideal manner, and may slip, loosen, or partially or completely open during use.

Some garments provide a pocket, flap or loop on the outside surface of the garment that may be used to stretch the waist without having to place a finger or thumb in such a place that it covers the fastener. Examples of such garments are disclosed in U.S. Pat. No. 6,213,991 to Kling et al., which is incorporated herein in its entirety and in a manner consistent with the present invention. Such garments may fail to prevent undesirable tensile loading of the fasteners because the loops or pockets may be located outboard of the fasteners where they create a tensile load in the fasteners when pulled.

Some garments may be provided with grips located along the laterally distal edges of the garment, beyond the fasteners, such as is disclosed, for example, in U.S. Pat. No. 5,624,429 to Long et al., which is incorporated herein by reference in its entirety and in a manner consistent with the present invention. These grips may be used to grasp the garment between finger and thumb without covering the fastener. Such garments may also fail to prevent the possibility of generating an unwanted tensile load in the fasteners because the grips are mounted outboard of the fasteners where they create a tensile load in the fasteners when pulled.

### SUMMARY OF THE INVENTION

It would be desirable to provide a garment that may be stretched without placing the fasteners under an undesirable tensile load. It would also be desirable to provide a garment that may be placed on a wearer without the individual placing the garment having to interfere with or otherwise deform the fasteners. It would further be desirable to provide a garment that enabled securing the fasteners to a target area prior to subjecting the fasteners to a tensile load. It also would be desirable to provide a method of making a garment that has fastener grips so that the garment may be stretched without placing the fasteners under an undesirable tensile load. The present invention is designed to overcome the foregoing and other deficiencies of prior art absorbent garments.

It is therefore a feature of an embodiment of the invention to provide a garment the may be stretched and placed on an individual without placing the fasteners of the garment under an undesirable tensile load. It is an additional feature of an embodiment of the invention to provide a method of making a garment by including fastener grips so that the garment may be stretched and placed on an individual without placing the fasteners under an undesirable tensile load. It is another feature of an embodiment of the invention to provide a method of placing a garment on an individual whereby the garment may be stretched during placement such that undesirable tensile forces are not created on the grips.

In accordance with these and other features of various embodiments of the invention, there is provided an absorbent garment having a topsheet, a backsheet, and an absorbent core disposed between the topsheet and the backsheet. The topsheet and the backsheet form a first waist region, a second waist region longitudinally opposite the first waist region, and a crotch region between the first and second waist regions.

A pair of tabs extend laterally away from opposite sides of the first waist region. The pair of tabs may be extensions of one or both of the topsheet and backsheet, or may be separate pieces of material. A fastener is attached to each tab, and one or more target devices are attached to the garment in the second waist region. The fasteners may be attached to the target devices so that the first waist region and second waist region of the garment are joined to one another to secure the garment on a wearer.

A pair of grips are attached to the garment in laterally opposite sides of the first waist region. Each grip is attached in an attachment area, which may have one or more attachment points selected to substantially evenly distribute a lateral tensile load on the grips throughout the first waist region. Each attachment area is located laterally inboard of the fastener on that side of the garment.

In accordance with another feature of an embodiment of the invention, there is provided a method of making an absorbent garment that includes preparing a topsheet, a backsheet, and an absorbent core, and disposing the absorbent core between the topsheet and the backsheet. The topsheet, backsheet, and absorbent core are prepared and arranged such that the topsheet and the backsheet form a first waist region, a second waist region longitudinally opposite the first waist region, and a crotch region between the waist regions. The method also includes preparing a pair of tabs and attaching them to the first waist region in such a manner that they extend laterally away from opposite sides of the first waist region.

The method also includes preparing a fastener and attaching the fastener to each tab, and preparing one or more target devices, and attaching the one or more target devices to the garment in the second waist region. The method also encompasses preparing a pair of grips, and attaching the pair of grips to the garment in laterally opposite sides of the first waist region, whereby each attachment area is located laterally inboard of the fastener on that side of the garment. Each grip is attached in an attachment area that may have one or more attachment points selected to substantially evenly distribute a lateral tensile load on the grips throughout the first waist region.

In accordance with another feature of an embodiment of the invention, there is provided a method of securing the garment described above to a subject that includes placing the crotch region of the garment over the crotch region of the subject. Placing the garment on the subject in this manner will result in the first waist region being adjacent either a front or back waist region of the subject, and the second waist region being adjacent the opposing waist region of the subject. The method then includes grasping the laterally opposing grips and pulling the laterally opposed sides of the first waist toward the second waist region in such a manner that lateral tensile loads on the grips are substantially evenly distributed throughout the first waist region. The method further includes attaching the fasteners that are attached on each laterally opposed tab to the one or more target devices disposed on the second waist region to thereby secure the garment to the subject.

These and other features of the invention will be readily apparent from the Detailed Description that follows, along with reference to the drawing appended hereto.

### BRIEF DESCRIPTION OF THE DRAWINGS

FIG. 1a is a partially cut-away view of an embodiment of the present invention, shown with elastic members fully stretched and with the grips slightly raised for clarity;

FIG. 1b is a plan view of a portion of an embodiment of the present invention, showing dimensional relationships;

FIG. 2a is a cross sectional view of the embodiment of FIG. 1a, as seen from reference line AA, in an unstretched condition;

FIG. 2b is a cross sectional view of the embodiment of FIG. 1a, as seen from reference line AA, in a stretched condition;

FIG. 3a is a detailed end view of one corner of an embodiment of the invention showing the construction of one tab and grip, wherein the grip is connected solely to the tab;

FIG. 3b is a detailed end view of one corner of an embodiment of the invention showing the construction of another tab and grip, wherein the tab is connected solely to the grip;

FIG. 3c is a detailed end view of one corner of an embodiment of the invention showing the construction of yet another tab and grip, wherein the grip is not connected to the tab;

FIG. 3d is a detailed end view of one corner of an embodiment of the invention showing the construction of another tab and grip;

FIG. 3e is a detailed end view of one corner of an embodiment of the invention showing the construction of yet another tab and grip, wherein the tab and grip are constructed from one piece of material;

FIG. 4 is the embodiment of FIG. 3c under a tensile load;

FIG. 5a is a plan view of one corner of an embodiment of the invention;

FIG. 5b is a plan view of one corner of another embodiment of the invention;

FIG. 5c is a plan view of one corner of yet another embodiment of the invention;

FIG. 5d is a plan view of one corner of another embodiment of the invention;

FIG. 6a is a detailed end view of the attachment between the first waist region and the second waist region of an embodiment of the invention;

FIG. 6b is a detailed end view of the attachment between the first waist region and the second waist region of the embodiment of FIG. 6a;

FIG. 7a is a detailed end view of the attachment between the first waist region and the second waist region of an embodiment of the invention;

FIG. 7b is a detailed end view of the attachment between the first waist region and the second waist region of the embodiment of FIG. 7a;

FIG. 8 is a partially cut-away view of an embodiment of the present invention having tabs formed by extension of the backsheet, shown with elastic members fully stretched and with the grips slightly raised for clarity;

FIG. 9 is a plot of the stretched position of fasteners attached to various types of tabs; and,

FIG. 10 is a partially cut away drawing of an embodiment of a grip, tab, fastener, and a portion of the remainder of an exemplary garment of the present invention.

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein.

The invention relates to a garment having a topsheet, a backsheet, and an absorbent core disposed between the topsheet and the backsheet. The topsheet and the backsheet form a first waist region, a second waist region longitudinally opposite the first waist region, and a crotch region between the first and second waist regions. A pair of tabs extend laterally away from opposite sides of the first waist region. The pair of tabs may be extensions of one or both of the topsheet and backsheet, or may be separate pieces of material. A fastener is attached to each tab, and one or more target devices are attached to the garment in the second waist region. The fasteners may be attached to the target devices so that the first waist region and second waist region of the garment are joined to one another to secure the garment on a wearer. In the absorbent garment, a pair of grips are attached to the garment in laterally opposite sides of the first waist region. Each grip is attached in an attachment area, which may have one or more attachment points selected to substantially evenly distribute a lateral tensile load on the grips throughout the first waist region. Each attachment area is located laterally inboard of the fastener on that side of the garment.

In one preferred embodiment of the invention, the fastener may be a hook portion of a hook and loop fastener, and the target area may be the loop portion of a hook and loop fastener. In another embodiment, the fastener may be an adhesive tape and the target devices may comprise a tape receiving surface. In another embodiment of the invention, a free edge of each grip, opposite that grip's attachment area, lies above the respective fastener when the grip is laid flat over the respective tab.

In various embodiments, the grip and tab may be connected to one another or to the first waist region. In one embodiment, the grips and the tabs may be constructed from opposite ends of pieces of material attached, along a central region thereof, to the garment at opposite sides of the first waist region. In a preferred embodiment, the grips may be used to attach each fastener to the one or more target devices without placing the fastener in tension. In yet another embodiment, each grip may have an anchoring fastener for holding the flap in a flattened position against the garment.

The invention now will be described with reference to the attached drawings illustrating preferred embodiments of the invention. For clarity, features that appear in more than one Figure have the same reference number in each Figure.

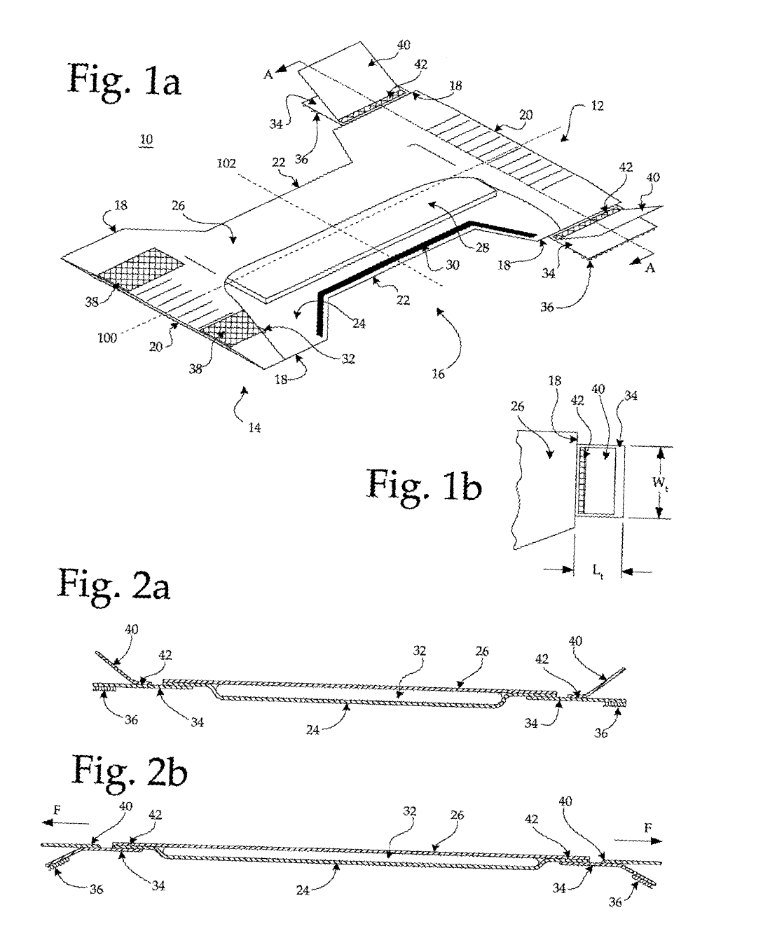

FIG. 1a is a partially cut away depiction of an exemplary embodiment of a garment 10 of the present invention. The embodiment shown in FIG. 1a is an infant's diaper, however, this depiction is not intended to limit the invention. The garment 10 of FIG. 1a is depicted in a generally flattened position, with the body-facing side facing down, and with the various elastic components depicted in their extended position for clarity. In the flattened position, the garment 10 may have a generally hourglass shaped structure, but it may also have any other shape suitable for the given application, such as a rectangular shape, a trapezoidal shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer.

The garment 10 preferably comprises a topsheet 24, and a backsheet 26, which may be substantially coterminous with the topsheet 24. When the garment 10 is being worn, the topsheet 24 faces the wearer's body, and the backsheet 26 faces away from the wearer. An absorbent core 28 preferably is disposed between at least a portion of the topsheet 24 the backsheet 26. An embodiment of the present invention may further comprise various additional features. One or more pairs of elastics gathers 30 may extend adjacent the crotch edges 22. The garment 10 may also comprise one or more waste containment systems, such as inboard standing leg gathers (not shown). One or both of the first and second waist regions 12, 14 may also be equipped with strips of elastic waist foam 32 or other elastically extensible material, which help contract the garment around the wearer's waist, providing improved fit and leakage prevention.

A pair of tabs 34 extend laterally away from opposite lateral edges 18 of the first waist region 12 of the garment 10. The tabs 34 may comprise an elastically extensible material, and may be designed to stretch around a wearer's waist to provide improved fit, comfort, and leakage protection. Such elasticized tabs 34 may be used in conjunction with, or in lieu of, waist foam 32, or other elastically extensible materials 32.

Referring now to FIG. 1b, in a preferred embodiment, the tabs 34 are relatively wide tabs. As understood herein, “wide” tabs are understood to include tabs 34 having a width Wt of greater than about 60 millimeters.

“Wide” tabs also are understood to encompass tabs 34 having a width to length ratio (Wₜ/Lₜ) of greater than about 0.6:1.0.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14.

A grip 40 is attached along one of its edges to the garment 10, preferably, but not necessarily, proximal to each tab 34. The grip attachment areas 42 preferably are located between the longitudinal centerline of the garment 10 and each respective fastener 36. When the garment 10 is affixed to a wearer, the caregiver may pull the grips 40 around the wearer and over the target devices 38 then secure the fasteners 36 to the one or more target devices 38. By pulling on the grips 40, and not on the ends of the tabs 34, the fasteners 36 may be isolated from the tensile load required to expand the elastics, and may be affixed without first placing them under a tensile load.

The various parts of the garment 10 are attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

These features, and other features, functions and uses of the present invention, are described in greater detail herein.

The topsheet 24 and backsheet 26 may be constructed from a wide variety of materials known in the art. The invention is not intended to be limited to any specific materials for these components. The topsheet 24 and backsheet 26 can be shaped and sized according to the requirements of each of the various types of absorbent garment, or to accommodate various user sizes. In an embodiment of the invention in which the garment 10 is a diaper or an adult incontinence brief, the topsheet 24, backsheet 26, or both, may have an hourglass shape, as seen in FIG. 1a, or may have a rectangular, trapezoidal, “T” shape, or other shape.

The backsheet 26 generally is made of any suitable pliable liquid impervious material known in the art or later discovered. Typical backsheet materials include films of polyethylene, polypropylene, polyester, nylon, and polyvinyl chloride and blends of these materials. For example, the backsheet 26 may be made of a polyethylene film having a thickness in the range of 0.02-0.04 mm. The backsheet 26 may be pigmented with, for example, titanium dioxide, to provide the garment 10 with a pleasing color or to render the backsheet 26 opaque enough that exudates being contained by the garment 10 are not visible from outside the garment. In addition, the backsheet 26 may be formed in such a manner that it is opaque, for example, by using various inert components in the polymeric film and then biaxially stretching the film. Other backsheet materials will be readily apparent to those skilled in the art. The backsheet 26 preferably should have sufficient liquid imperviousness to prevent any leakage of fluids through the backsheet 26. The required level of liquid imperviousness may vary between different locations on the garment 10.

The backsheet 26 may further comprise separate regions having different properties. In a preferred embodiment, portions of the backsheet 26 are air-permeable to improve the breathability, and therefore comfort, of the garment 10. The different regions may be formed by making the backsheet 26 a composite of different sheet materials, chemical treatment, heat treatment, or other processes or methods known in the art. Some regions of the backsheet 26 may be fluid pervious. In one embodiment of the invention, the backsheet 26 is fluid impervious in the crotch 16, but is fluid pervious in portions of the first and second waist regions 12, 14. The backsheet 26 may also be made from a laminate of overlaid sheets of material.

The backsheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with the present application and invention. Materials for such a fibrous outer liner include a spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers.

The moisture-pervious topsheet 24 may be made of any suitable relatively liquid-pervious material currently known in the art or later discovered that permits passage of a liquid therethrough. Examples of suitable topsheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 28. The topsheet 24 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a topsheet material for the given application. The present invention is not intended to be limited to any particular material for the topsheet 24, and other topsheet materials will be readily apparent to those skilled in the art.

The topsheet 24 may further comprise several regions having different properties. In one embodiment of the present invention, the laterally distal portions of the topsheet 24 are preferably substantially fluid impervious and hydrophobic, while the remainder of the topsheet 24 is hydrophilic and fluid pervious. Different topsheet properties, such as fluid perviousness and hydrophobicity, may be imparted upon the topsheet 24 by treating the topsheet 24 with adhesives, surfactants, or other chemicals, using a composite of different materials, or by other means. The topsheet 24 may also be made from a laminate of overlaid sheets of material. The topsheet 24 also may be treated in specific areas like the crotch region, with skin wellness ingredients such as aloe, vitamin E, and the like.

As noted elsewhere herein, the topsheet 24 and backsheet 26 may be substantially coterminous, or they may have different shapes and sizes. The particular design of the topsheet 24 and backsheet 26 may be dictated by manufacturing considerations, cost considerations, and performance considerations. Preferably, the topsheet 24 is large enough to completely cover the absorbent core 28, and the backsheet 26 is large enough to prevent leakage from the garment 10. The design of topsheets 24 and backsheets 26 is known in the art, and a skilled artisan will be able to produce an appropriate topsheet 24 and an appropriate backsheet 26 without undue experimentation.

The topsheet 24 and the backsheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the topsheet 24 to the backsheet 26. The particular joining method may be dictated by the types of materials selected for the topsheet 24 and backsheet 26.

An absorbent core 28 preferably is disposed between the topsheet 24 and the backsheet 26 in at least the crotch region 16. The absorbent core 28 may extend into either or both of the first and second waist regions 12, 14. Although the absorbent core 28 depicted in FIG. 1a has a substantially rectangular shape, other shapes may be used, such as a “T” shape or an hourglass shape. The shape of the absorbent core 28 may be selected to provide the greatest absorbency with a reduced amount of material. The absorbent core may be associated with the topsheet 24, backsheet 26, or any other suitable part of the garment 10 by any method known in the art, in order to fix the absorbent core 28 in place.

The absorbent core 28 may be made from any suitable material or materials known in the art that are capable of absorbing or retaining liquids (e.g., urine and/or menses). Examples of suitable materials for use as the absorbent core 28 include creped cellulose wadding, absorbent foams, absorbent sponges, super absorbent polymers, absorbent gelling materials, fiberized cellulose, chemically stiffened, modified or cross-linked cellulose fibers, fluff pulp having tissue or synthetic materials between the absorbent core 28 and the topsheet 24, comminuted wood pulp (commonly known as “airfelt”) or any equivalent material or combination of materials. The size and capacity of the absorbent material should correspond to the application, for example, an incontinent brief for an adult may require a larger absorbent core than a diaper for a child. Zoned absorbency may also be used, if desired. For example, more absorbent capacity may be located in particular regions of the garment 10 depending on the gender of the intended wearer. The invention is not intended to be limited to any specific materials for use in the absorbent core 28.

In a preferred embodiment, the absorbent core 28 comprises super absorbent polymer distributed within a fibrous structure. Absorbent cores of this type are known in the art, and exemplary absorbent cores are described in U.S. Pat. No. 5,281,207, issued to Chmielewski et al., and U.S. Pat. No. 5,863,288, issued to Baker, the disclosures of each of which are herein incorporated by reference in their entirety.

Additional sublayers, transfer layers, acquisition layers, tissue wraps, and the like also may be incorporated into the absorbent core 28. Such layers may be provided to assist with transferring fluids to the absorbent core 28, handling fluid surges, preventing rewet, containing absorbent material, improving core stability, or for other purposes. For example, a substantially rectangular, preferably nonwoven, sublayer (not shown), having a basis weight of about 0.1-2 oz., preferably about 0.4-0.6 oz., may overlay absorbent core 28. Those skilled in the art are capable of selecting materials, dimensions, and locations for such layers without undue experimentation.

In a preferred embodiment, the absorbent core 28 is a laminate comprised of a layer of superabsorbent polymer material in the form of particles disposed between two air-laid tissues, first and second tissue layers (or “upper” and “lower” tissue layers). The first and second tissue layers contain the superabsorbent polymer material, improve lateral wicking of the absorbed exudates throughout the absorbent core 28 and provide a degree of absorbency.

The crotch region 16, or other regions of the garment 10, may also include mechanical sealing devices to provide the garment 10 with a leak-proof fit around the wearer. In a preferred embodiment, the crotch edges 22 each have one or more elastic leg gathers 30 placed along them to help the crotch edges 22 contract about the contours of the wearer's body, thereby providing a leak-resistant seal. Such elastic leg gathers 30 are known in the art, and are disclosed, for example, in U.S. Pat. No. 5,830,203, issued to Suzuki et al., the disclosure of which is herein incorporated by reference in its entirety in a manner consistent with the present invention. The elastic leg gathers 30 may extend into the first waist region 12, and may extend as far as the front longitudinally distal edge 20.

In another preferred embodiment, standing leg gathers (not shown) are disposed on the topsheet 24. Standing leg gathers are strips of material that rise vertically from the surface of the topsheet 24 to provide additional sealing to the garment 10. The standing leg gathers may extend across all or part of the garment 10 along its longitudinal axis 100. Typically, one standing leg gather is located on either side of the absorbent core 28. The standing leg gathers may be made from folded portions of the topsheet 24 or backsheet 26, or may be made from additional strips of material. Each standing leg gather may be equipped with one or more elastic elements to help seal the gather to the wearer's body. Standing leg gathers are known in the art, and disclosed in U.S. Pat. No. 5,292,316, issued to Suzuki, the disclosure of which is herein incorporated by reference in its entirety in a manner consistent with the present invention.

Any suitable elastic material may be used for the elastic leg gathers 30 and the standing leg gathers. Preferably, the elastic material can be stretched to between at least 10% to 300% of its original length without losing its resilience. The elastic material used for the elastic leg gathers 30 and standing leg gathers may, for example, comprise an elastic film, a multidirectional elastic aggregate such as elastic webbing, netting, or scrim elastic, or strands or bands of suitable elastic materials, such as natural or synthetic rubber, urethane elastomers, spandex, LYCRA and elastic polymers. Other suitable elastics will be apparent to those skilled in the art in light of the present teachings. The elastics materials may be attached to the garment 10 in any of several ways known in the art. For example, the elastic materials may be ultrasonically bonded, heat/pressure sealed using a variety of bonding patterns, or glued to the diaper 10 using a variety of adhesives. Other performance enhancing devices, such as pockets, baffles, and openings in the topsheet also may be used with the present invention.

The first and second waist regions 12, 14 may be joined to one another by a pair of fasteners 36, or a plurality of fastening elements 36, each of which is attached to a tab 34 protruding from an opposite lateral side edge 18 of the first waist region 12. The fasteners 36 may interact with one or more target devices 38 located in the second waist region 14 to provide sufficient gripping force to hold the garment 10 on a wearer. While the embodiment depicted in FIG. 1a shows the tabs 34 and fasteners 36 being located in the first waist region 12, and the target device 38 being in the second waist region 14, skilled artisans will recognize that these locations may be reversed. Exemplary embodiments of the tabs 34, fasteners 36 and target devices 38 are provided herein, however, the present invention is not intended to be limited to any particular construction or materials for these parts.

The tabs 34, fasteners 36, and target devices 38, preferably are located and sized so that they may be used to wrap the first and second waist regions 12, 14 around wearers having a variety of sizes. For example, additional elastics (not shown) may be added to the garment 10 in one or both of the first and second waist regions 12, 14 in order to increase the range of user sizes that may be fitted by a single size garment 10. In a preferred embodiment, a strip of elastic foam 32 is located in each waist region to provide the waist regions with elasticity, additional comfort, and leakage prevention. The strips of elastic foam 32, such as an elastic polyurethane foam, also may help the longitudinally distal edges 20 to resist doubling over (“rolling”) during use.

The tabs 34 preferably are attached to the garment 10 at opposite lateral edges 18 of the first waist region 12, and extend distally away from the center of the garment 10. The tabs 34 may be attached to the garment 10 using any method that provides a suitably strong bond. The tabs 34 may be attached in an edge-to-edge fashion with the garment, or the tabs 34 may overlap the topsheet 24 or backsheet 26, or another part of the garment 10, in order to provide a stronger or larger bond area. An overlapping arrangement is depicted in FIG. 2, which is a cross sectional view of the embodiment of FIG. 1a, as seen from reference line AA. Preferably, the tabs 34 are attached to one or both of the backsheet 26 and the topsheet 24. Exemplary methods for attaching the tabs 34 to the garment 10 include: ultrasonic welding, thermal bonding, adhesive bonding, stitching, stapling, combinations of the foregoing, and the like. A skilled artisan will be able to attach the tabs 34 to the garment 10 without undue experimentation.

The tabs 34 may comprise any suitable material. Nonwoven materials, such as those described elsewhere herein, may be particularly suitable for making the tabs 34. In a preferred embodiment, the tabs 34 comprise an elasticized material, such as a laminate of nonwoven sheets and elastic material. Also in a preferred embodiment, the tabs are comprised of a material that is easily bonded to the garment 10 and to the fasteners 36 by any of various means known in the art.

The fasteners 36 may comprise an adhesive tape, a portion of a mechanically interlocking fastener, such as the hook or loop portion of a hook and loop fastener, or a button, snap, or any other suitable fastening mechanism known in the art. Preferably, the fasteners 36 may be sealed and released numerous times without losing their holding and/or fastening ability. The target device 38 may be associated with a surface of the garment 10, or it may be an integral part of the surface of the garment. That is, the surface of the garment may itself act as a target device that will suitably interact with the fasteners 36. The selection and manufacture of fasteners 36, and target devices 38 is known in the art, and a skilled artisan will be able to implement such structures and devices without undue experimentation.

In a preferred embodiment, the fastener 36 is the hook portion of a hook and loop fastener. The fastener 36 may comprise a separate strip of material having a backing sheet with the hooks protruding therefrom, or the fastener 36 may protrude directly from the surface of each tab 34. The fasteners 36 also may protrude beyond the laterally distal edges of the tabs 34. The design and construction of such tabs 34 and fasteners 36 are disclosed, for example, in U.S. Pat. No. 5,624,429 to Long et al., the disclosure of which is incorporated herein by reference in its entirety and in a manner consistent with the present invention.

Also in a preferred embodiment, the target device 38 is the loop portion of a hook and loop fastener that is selected to mechanically engage with the fastener 36 to provide a secure, yet releasable and resealable bond. The target device 38 may be provided as a separate strip of material having a backing sheet with the loops protruding therefrom. In such a case, the target device or devices 38 may be attached to the outermost layer of the garment by any suitable means. In another embodiment, the target device 38 may also comprise a portion of the outer surface material of the garment (whether the outer surface is the backsheet 26 or an outer cover, as discusses elsewhere) that can mechanically engage with the fasteners 36, or that is mechanically or chemically treated to be able to mechanically engage with the fasteners 34.

In any case in which the fasteners 36 or target device 38 may cause irritation of the wearer's skin, it is preferred that such fasteners 36 or target devices 38 be shielded from contact with the wearer, such as by covering the target or fastener 38, 36 with a protective cover prior to being fastened together. Such a protective cover may also serve to prevent the target devices or fasteners from becoming contaminated and inoperable.

Grips 40 preferably are attached to the garment 10, one in proximity to each tab 34. Each grip 40 is attached, along at least one of its edges, to the garment 10 in an attachment area 42 such that the free end of the grip 40 extends away from the wearer. Each attachment area 42 is located either at the longitudinal centerline 100, or between the longitudinal centerline 100 of the garment 10 and the fastener 36, as can be seen in FIGS. 1 and 2 (i.e., inboard of the fasteners 36). The attachment points may be on the backsheet 26, the topsheet 24, the tabs 34 or any other part or combination of parts of the garment 10 in the first waist region 12. The grips may comprise any material that may be joined to either the tabs 34 or an outer or inner surface of the garment in the first waist region 12. Suitable materials include nonwoven materials, as described elsewhere herein, non-woven spun bonded or carded webs of polypropylene, polyethylene, nylon, polyester, and blends of these materials, or other fabric materials, films, and the like.

When caregivers place absorbent garments on wearers, they often must pull directly on the fasteners of those garments in order to stretch the garment around the wearer. The tensile force thus created often is maintained by the caregiver when attaching the fastener to the garment. This tensile force may cause the fastener to deform, as some parts of the fastener may be under greater tension than others, and this deformation may cause the fastener to grip less firmly than it normally can grip. For example, in a typical case, a caregiver pulls on the fastener by gripping it between the thumb and fingers and attaches the portion of the fastener not covered by the thumb or fingers to the garment. The caregiver then removes the thumb or fingers and attaches the remainder of the fastener to the garment. The portion first attached to the garment may slip in the interim, or the caregiver may pull with a different force, or not pull at all, when attaching the portion originally covered by the thumb or fingers, possibly causing a differential gripping condition between the two portions and weakening the grip. In other cases, the caregiver may simply omit the step of attaching the portion covered by the thumb or finger, or may only attach those regions of the fastener 36 that are near the thumb or fingers, which also causes less than optimal gripping force.

While not intending on being bound by any theory, the present inventor has been found that the problems associated with attaching conventional fasteners 36 to the garment 10 are exacerbated when wide tabs 34 are used, and particularly when elasticized wide tabs 34 are used. The generally greater size of wide tabs 34 appears to allow a greater degree of distortion in the fasteners 36 when a tensile load is applied to the tabs 34 in a conventional manner (i.e., by holding the tabs or fasteners between the fingers and thumb). For example, a caregiver typically holds a tab 34 (or fastener 36) at or near the middle of its width, Wₜ. When the caregiver extends the tab 34, the inventor believes that the tensile force is concentrated in the center of the tab 34, stretching that portion of the tab more than the unheld portions. The concentrated force is redistributed to the other portions of the tab 34, however, and those portions experience some stretch, but the amount of stretch may be substantially less in the unheld portions than the held portion. The amount of tensile load redistribution, and thus the amount of stretch in the unheld portions of the tab 34, depend on several variables, including the width and stiffness of the tab 34. Narrower tabs 34 and stiffer tabs 34 generally redistribute the concentrated force more quickly (i.e., within a relatively short physical distance from the concentrated load created by the caregiver's fingers and thumb), and thus the different portions of the fastener 36 tend to be attached with less distortion relative to more flexible (e.g., elasticized) tabs 34 and wider tabs 34.

FIG. 9 illustrates how a wider, more flexible tab 34 generally will be attached with a more distorted shape. In FIG. 9, the lines labeled C and B represent the final stretched position for conventional tabs 34 that have been stretched, at their middle, to the same distance for example, all the way to target devices 38. Line C represents a wider, more flexible tab, line B represents a narrower, more rigid tab. In each case, the tab 34 has been extended without the benefit of the present invention. Line A represents a wider, more flexible tab that has been associated with a grip 40 of the present invention. FIG. 9 shows that the tab 34 positioned using the grip 40 of the invention (e.g., line A) is more evenly attached across its width Wt than the other tabs. The operation of the grips 40 is described in more detail as follows.

The grips 40 of the present invention may be used to assist in laterally stretching the garment 10 without placing the fasteners 36 under a tensile load, thereby reducing the likelihood that the fastener 36 will provide inadequate gripping force. This is possible because the attachment areas 42 are located inboard of the fasteners 36, taking the fasteners 36 out of the direct path of the stretching force. A depiction of the lateral stretching force F, and the consequent deformation of the garment, is shown in FIG. 2b. As can be seen in FIG. 2b, the fasteners are not in the path of the stretching force F, and thus not under any substantial tensile load.

The grips 40 may be associated with the garment 10 at any location that allows them to be used to extend the tabs 34 around the waist of a wearer, provided that they are inboard of the fasteners 36. Inboard in the context of the present invention denotes a position closer to longitudinal centerline 100 than fasteners 36. Preferably, the grips are located such that they may be used to extend any elastic material in the first waist region 12, and the adjacent portions of the crotch region 16, such as by locating the grips 40 outboard of a waist elastic 32.

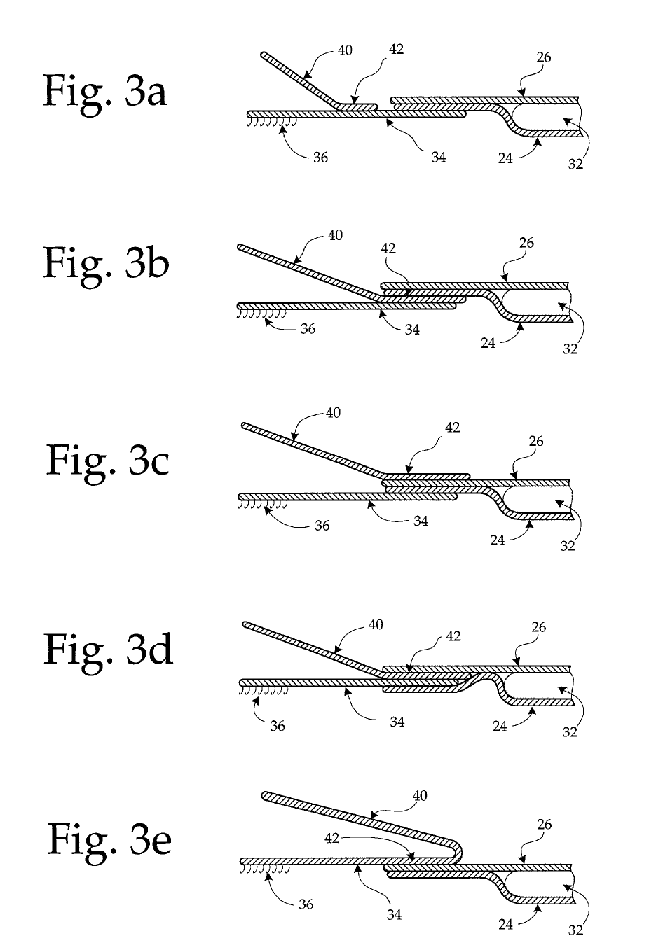

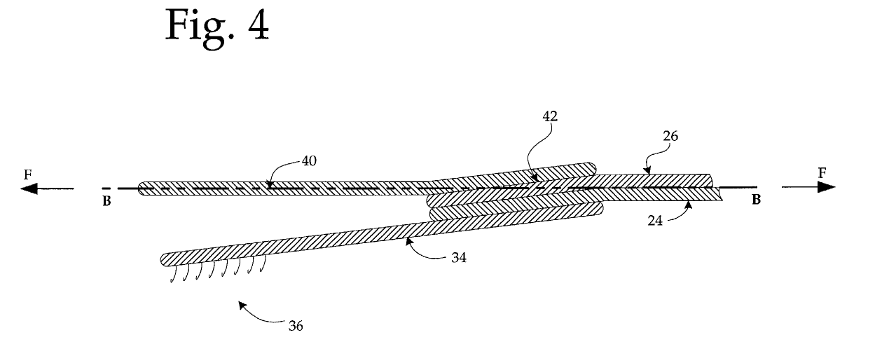

FIGS. 3a through 3e are detailed end-views of one side of preferred embodiments of the garment that show the spatial relationship between the various parts of the garment 10. In one embodiment of the invention, depicted in FIG. 3a, the grips 40 are attached to the tabs 34, and not to the topsheet 24 or the backsheet 26. In another embodiment, shown in FIG. 3b, the grips 40 are sandwiched between the tabs 34 and the topsheet 24, and operatively associated with both. In such an embodiment, the tabs 34 may or may not be joined directly to the topsheet 24 or backsheet 26. In another embodiment, shown in FIG. 3c, the grips 40 are associated with the backsheet 26, and the tabs 34 are associated with the topsheet 24. In yet another embodiment, the grips 40 and the tabs 34 may be sandwiched between the topsheet 24 and the backsheet 26, as shown in FIG. 3d. In still another embodiment of the present invention, shown in FIG. 3e, the grips 40 may be fabricated from the same piece of material as the tab 24. In each embodiment, the attachment area 42 between the grip 40 and the garment 10 is inboard of the fastener 36. Other configurations will be obvious to one skilled in the art based on the teachings provided herein, and the present invention is not limited to these embodiments.

Although these figures depict the grips 40 or the tabs 34 being operatively associated with either or both of the topsheet 24 and backsheet 26, it should be understood that the grips 40 and the tabs 34 may be attached to intermediate parts of the garment 10, such as a backsheet cover or other such parts.

The particular configuration of the tabs 34 and the grips 40 may be selected to provide certain benefits. For example, the configurations shown in FIGS. 3b, 3c, and 3d may provide relatively inexpensive manufacturing costs because they may require fewer attachment operations or for other reasons. The embodiment of FIG. 3a, on the other hand, may require additional bonds, but it may be less expensive to provide the grips 40 and the tabs 34 as a separate assembly during the assembly process, thereby costing less overall.

Another factor to consider when selecting the bond configuration is how deformation of the topsheet 24, backsheet 26, and grips 40 under a tensile load will affect the orientation of the tabs 34 and, consequently, the fasteners 36. FIG. 4 depicts the embodiment of FIG. 3c under a tensile load F. When a tensile load is applied to the grips 40, the grips 40 will tend to become aligned generally in a substantially straight line with the central planar axis BB of the topsheet 24 and backsheet 26. (As understood herein, the central planar axis BB of the topsheet 24 and backsheet 26 is the axis defined by a plane located between and parallel with the topsheet 24 and backsheet 26.) If the grips 40 are attached to the garment 10 towards the outside of the garment relative to the central planar axis BB of the topsheet 24 and the backsheet 26, then the tensile force F generally causes the tabs 34 and the fasteners 36 to point toward the inside of the garment 10 when a tensile load is applied, as is shown in FIG. 4. If the grips 40 are in line with the central planar axis BB, as in the embodiment of FIGS. 3a and 3d, then the tabs 34 and the fasteners 36 generally tend to stay flat (i.e., not move towards the inside or the outside of the garment 10) when the grips 40 are under a tensile load F. If the grips 40 are inside the central planar axis BB, then the tabs 34 and fasteners may tend to move towards the outside of the garment 10, and press against the grip 40 when the tensile load F is applied.

In a preferred embodiment, the grips 40 are attached to the garment 10 such that they are in line with the central planar axis BB of the backsheet 26 and topsheet 24, or inside the central planar axis BB. In this embodiment, the tabs 34 and the fasteners 36 tend to move away from the wearer while the garment is being attached, which may prevent irritation caused by contact between the wearer's skin and the tabs 34 or the fasteners 36. In addition, in such an embodiment, the tabs 34 and the fasteners 36 may press against the backs of the caregiver's fingers, allowing the caregiver to accurately guide the fasteners to the target devices 38. Once the fasteners 36 are placed over the target devices 38, the backs of the caregiver's fingers may be used to press the fasteners 34 into place while still maintaining the tensile load on the grips 40, thereby allowing the fasteners 36 to be engaged while in an unstressed condition. Such an embodiment may also help prevent the fasteners 36 from attaching to or interfering with other parts of the garment 10 or other objects while the grips are being manipulated.

The grips 40 may be made from a variety of materials. For example, the grips 40 may be made from sheets of woven or nonwoven material, or from films. For convenience and cost reduction, the material preferably is one that may be easily and inexpensively bonded with the part of the garment 10 to which the grips 40 are desired to be attached, such as the backsheet 26 or tabs 34. The grip material also may be selected to have a high axial stiffness. Axial stiffness is the measure of a material's ability to resist in-plane deformation, such that it tends not to stretch when placed under a tensile load. When axially rigid materials are used for the grips 40, the tensile forces are transmitted more directly to the elastic materials in the remainder of the garment 10 because the grips 40 themselves will not extend appreciably under the load. Furthermore, the grip material may also be selected to exhibit a relatively high degree of bending stiffness (i.e., the ability to resist out-of-plane deformations) compared with the tabs 34 and other materials used in the garment 10. Tabs 40 comprising materials having a combination of bending stiffness and axial stiffness tend to resist in-plane and out-of-plane deformations, thereby helping to stiffen the garment along the attachment points 42. This additional stiffness may be useful for conveying tensile forces in the grips 40 directly to the garment 10 or tabs 34, and isolating the portions of the tabs 34 having the fasteners 36 from the tensile forces. The added bending stiffness may also prevent the grips 40 from deforming, which may cause the tabs 34 and the fasteners 36 to deform during attachment. In addition, this added stiffness may help prevent the longitudinally distal edges 20 from doubling over during use (also known as “rolling”).

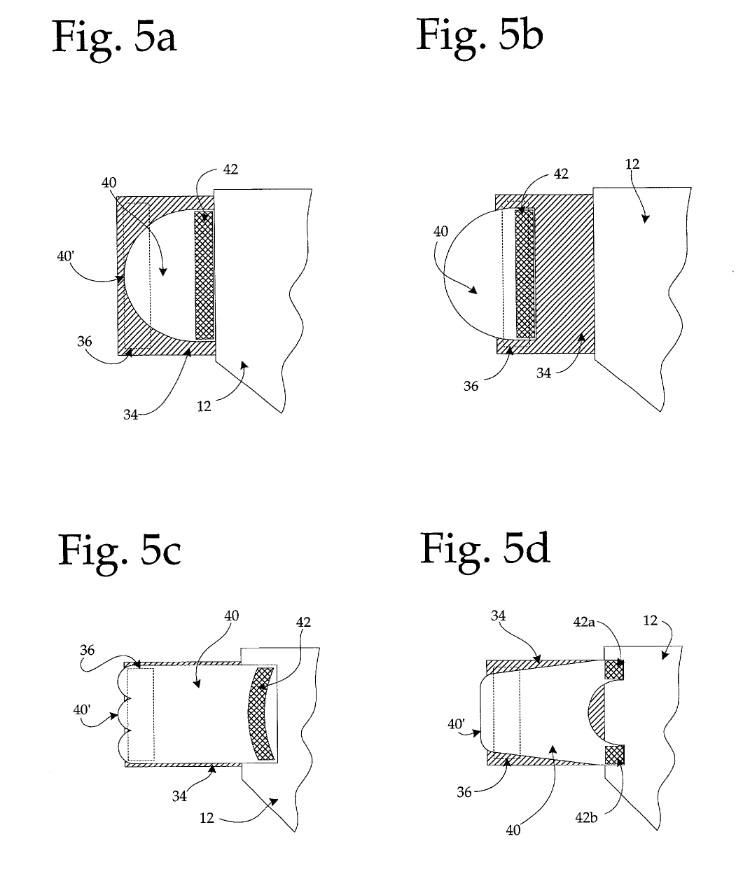

The grips 40 may be made to have any shape. Exemplary shapes are provided in FIGS. 5a through 5d. Ideally, the grips are shaped such that they are large enough to be easily grasped and held while being used to affix the garment 10 to a wearer, yet small enough that they will tend to stay close to the garment 10 during use and not interfere with clothing or other objects. In addition, if the grips 40 are constructed of material having a relatively high bending stiffness, they may tend to press flatter against the garment. In a preferred embodiment, such as those depicted in FIGS. 5a, 5c, and 5d, the grip free ends 40′ are substantially coterminous with the location of the fastener 36 (shown as a broken line). In these embodiments, the caregiver may grasp each grip 40 between the thumb and fingers and apply pressure against the fastener 36 with the backs of the fingers or thumb to affix each fastener 36 to the associated target device 38 with the use of one hand.

The grips 40 may be associated with the garment 40 by any method known in the art, and the available methods may vary depending on the particular materials that are selected for the various parts. Exemplary joining methods include ultrasonic bonding, joining with hot melt adhesives or other chemical, thermal, or thermochemical bonding methods, mechanical fastening, such as by stitching, stapling, pinning, and the like, and any other suitable joining methods or devices.

The grips 40 may be joined to the garment 10 in such a manner so as to provide a more or less direct tensile force on the various elastic members of the garment 10. For example, in the embodiment of FIG. 5d, a first attachment area 42a is located such that it concentrates tensile forces in the region of the waist elastic 32 and other elastics located in the first waist region 12. A second attachment area 42b concentrates tensile forces in the ends of the elastic leg gathers 30 or other elastics present at or near the crotch region 16. By providing a more direct path between the grip 40 and the elastics, the garment 10 may be more evenly stretched while putting the garment 10 on a wearer.

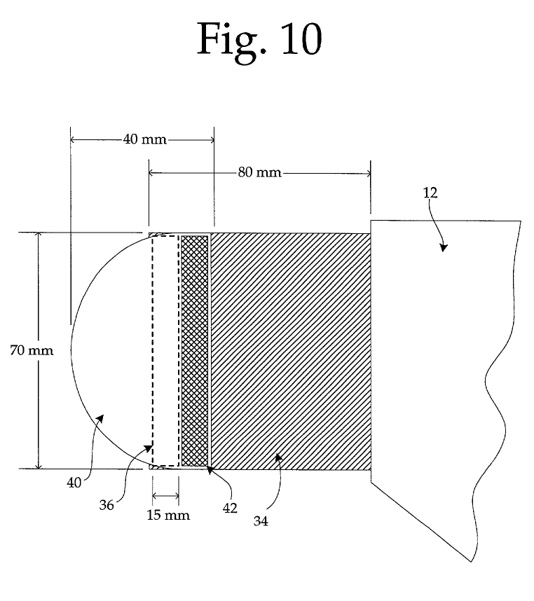

It has been found that grips 40 and tabs 34 having the dimensions of the following exemplary embodiment have provided desirable results with an infant diaper having a width (not including the grips and tabs) of between about 220 and 300 millimeters. In an exemplary embodiment, depicted in FIG. 10, each tab 34 extends about 70 millimeters in the longitudinal direction 100 of the garment, and about 80 millimeters laterally from the lateral edge 18 of the garment. Each fastener 36 extends about 70 millimeters longitudinally and about 15 millimeters laterally, and is attached to one side of the distal end of the respective tab 34. Each grip 40 is attached the respective tab 34 at a bond area 42 that is just inboard of the respective fastener 36. Each grip 40 extends about 70 millimeters longitudinally and extends laterally outboard from the attachment area 42 by a distance of about 40 millimeters. In this exemplary embodiment, the grips 40 may be attached to the tabs 34 by adhesive bonding, ultrasonic bonding, heat bonding, and the like. In one embodiment, a combination of bonding methods, such as adhesive bonding reinforced with ultrasonic bonding, may be used.

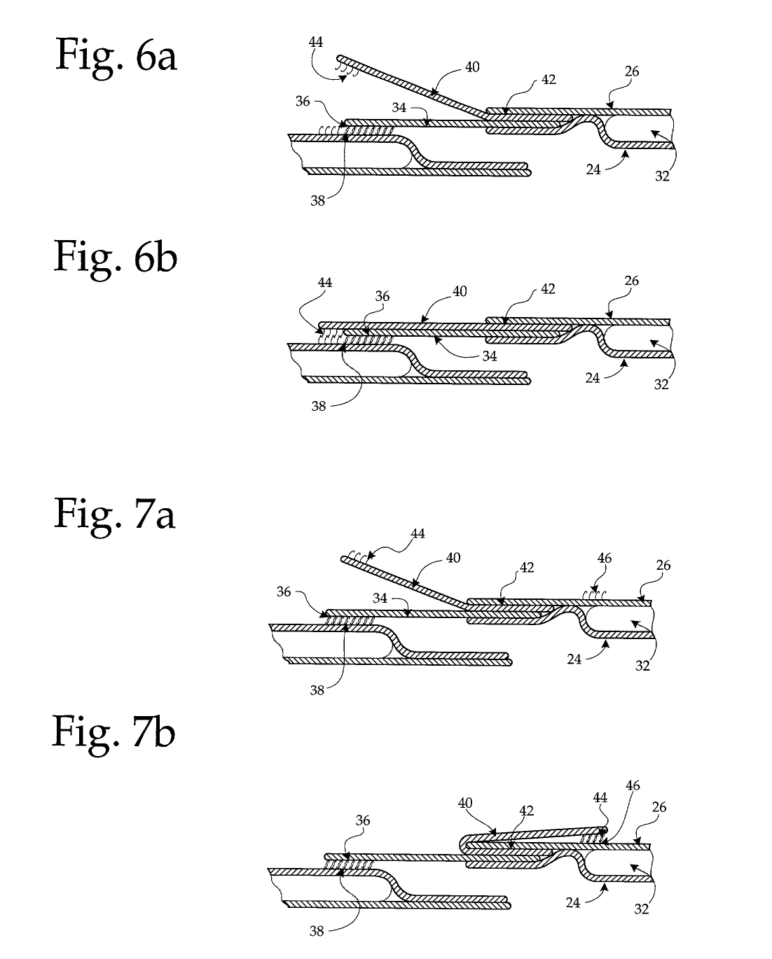

Referring now to FIGS. 6a, 6b, 7a and 7d, the grips 40 also may be equipped with anchoring fasteners 44 to hold the grips 40 against the surface of the garment 10. the anchoring fasteners 44 may comprise any suitable fastener, as described elsewhere herein with regard to the fasteners 36.

Such anchoring fasteners 44 may be desirable to prevent the grips 40 from interfering with or becoming hooked on clothing and other objects. FIG. 6a depicts an embodiment of the invention in which the anchoring fasteners 44 are located on the same side of each grip 40 as the tab 34. In such as case, as may be seen from FIG. 6b, the anchoring fasteners 44 may be selected to attach to the target device 38, or to another mating surface located on the second waist region 14 or the tab 34.

In another embodiment, the anchoring fasteners 44 may comprise an adhesive or other attachment surface on the side of the each grip 40 facing the tab 34. In such a case, the anchoring fasteners 44 may further comprise a protective layer (not shown), such as a silicone or plastic sheet. The protective layer prevents the anchoring fasteners 44 from attaching to the tabs 34 until the grips 40 have been used to attach the fasteners 36. Once the fasteners 36 have been attached, and the grips 40 are no longer necessary, the protective layer may be removed and the grips 40 adhered to the surface of the tabs 34.

The anchoring fasteners 44 also may be attached to the surface of each grip 40 facing away from the tab 34, as is depicted in FIGS. 7a and 7b. In such an embodiment, backsheet 26 or other outer layer of the garment 10 may be provided with additional target surfaces 46 for engaging with the anchoring fasteners 44. The anchoring fasteners 44 may also be selected to engage directly with the outer surface of the garment 10.

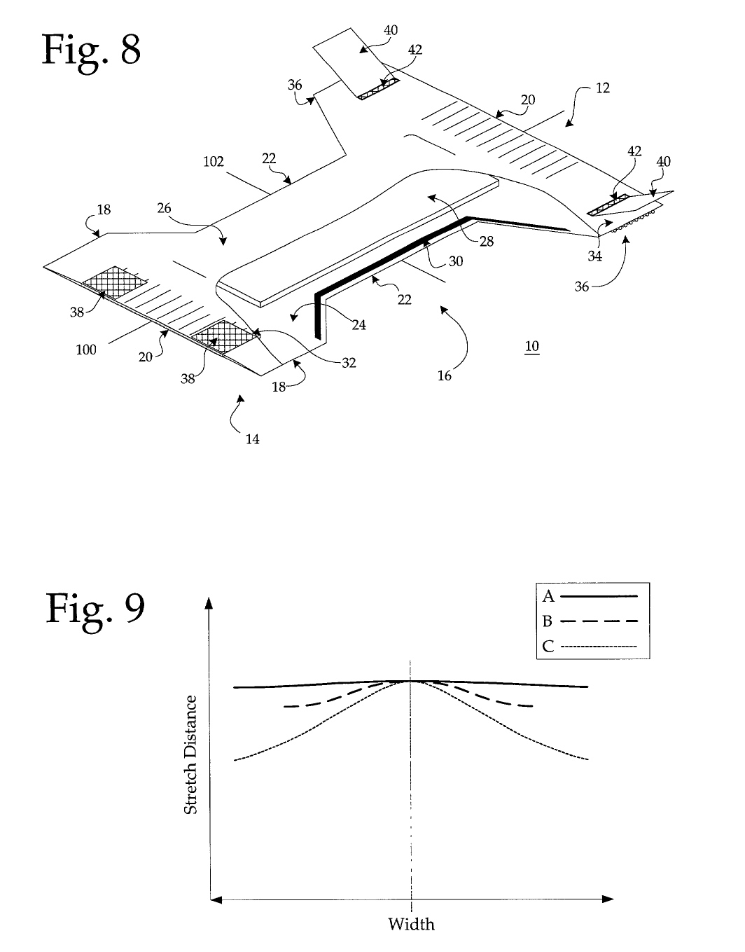

The embodiments described thus far have all involved a garment 10 having a pair of tabs 34 to which the fasteners 36 are attached. The present invention may also be used in an embodiment in which the tabs 34 are extensions of one or both of the topsheet 24 and backsheet 26. In such a case, the fasteners 36 may be attached directly to the topsheet 24 or other suitable part of the garment 10. Such an embodiment is depicted in FIG. 8.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

I claim:

1. An absorbent garment having a longitudinal dimension and a lateral dimension comprising:
a topsheet;
a backsheet, whereby the topsheet and the backsheet form a first waist region, a second waist region longitudinally opposite the first waist region, and a crotch region therebetween;
an absorbent core disposed between the topsheet and the backsheet;
a pair of tabs, each tab extending laterally away from an opposite lateral side of the first waist region;
at least one fastening element attached to each tab;
one or more target devices attached to the garment in the second waist region, where the at least one fastening element and the one or more target devices are capable of attaching to one another, the one or more target devices being located so that the first waist region and second waist region of the garment may be joined to one another to secure the garment on an intended wearer; and
a pair of grips, each grip being attached at an attachment area on laterally opposite sides of the garment in the first waist region, whereby each attachment area is laterally inboard of at least one fastening element attached to each respective tab.

2. The absorbent garment of claim 1, wherein each tab comprises a portion of at least one of the topsheet and backsheet.

3. The absorbent garment of claim 1, wherein the tabs comprise separate pieces of material attached to the garment at or near opposite lateral side edges of the first waist region.

4. The absorbent garment of claim 1, wherein the at least one fastening element comprises a hook portion of a hook and loop fastener and the one or more target devices comprise the loop portion of a hook and loop fastener.

5. The absorbent garment of claim 1, wherein the at least one fastening element is an adhesive tape and the one or more target devices comprise a tape receiving surface.

6. The absorbent garment of claim 1, wherein a free edge of each grip opposite the respective attachment area is proximal to the respective at least one fastening element when the grip is laid flat over the respective tab.

7. The absorbent garment of claim 1, wherein each grip is attached only to the respective tab.

8. The absorbent garment of claim 1, wherein each grip is attached to at least one of the topsheet and the backsheet.

9. The absorbent garment of claim 1, wherein each grip is attached to one of the topsheet and the backsheet and each tab is attached only to the respective grip.

10. The absorbent garment of claim 1, wherein the pair of grips are used to attach each of the at least one fastening elements to the one or more target devices without placing the fastening elements in tension.

11. The absorbent garment of claim 1, wherein each grip and its respective tab comprise opposite portions of a piece of material attached to the garment in a central region of the piece of material.

12. The absorbent garment of claim 1, wherein each grip further comprises an anchoring fastener for holding the grip in a flattened position against the garment.

13. The absorbent garment of claim 1, wherein each attachment area comprises one or more attachment points located such that a lateral tensile load on the grips will be substantially evenly distributed throughout the first waist region.

14. The absorbent garment of claim 1, wherein the tabs are elasticized tabs.

15. The absorbent garment of claim 1, wherein the tabs are relatively wide tabs.

16. The absorbent garment of claim 1, wherein the tabs are relatively wide elasticized tabs.

17. An absorbent garment having a longitudinal dimension and a lateral dimension comprising:
a topsheet;
a backsheet overlaying the topsheet;
the topsheet and the backsheet forming a first waist region, a second waist region longitudinally opposite the first waist region, and a crotch region therebetween;
an absorbent core disposed between the topsheet and the backsheet;
a means for attaching the first waist region to the second waist region to form a pant-like garment; and
a means for laterally extending the garment without creating a tensile force in the means for attaching the first waist region to the second waist region.

18. The absorbent garment of claim 17, wherein the means for attaching comprises a mechanical fastening mechanism.

19. The absorbent garment of claim 17, wherein the means for attaching comprises a hook and loop fastener.

20. The absorbent garment of claim 17, wherein the means for attaching comprises a tape fastener.

21. The absorbent garment of claim 17, further comprising:
a pair of tabs extending laterally away from opposite lateral sides of the first waist region;
wherein at least a first portion of the means for attaching is disposed on each tab.

22. The absorbent garment of claim 21, wherein a second portion of the means for attaching is disposed on the second waist region.

23. The absorbent garment of claim 21, wherein the pair of tabs comprise portions of at least one of the topsheet and the backsheet.

24. The absorbent garment of claim 21, wherein the means for extending is attached to the pair of tabs.

25. The absorbent garment of claim 21, wherein the tabs are elasticized tabs.

26. The absorbent garment of claim 21, wherein the tabs are relatively wide tabs.

27. The absorbent garment of claim 21, wherein the tabs are relatively wide elasticized tabs.

28. The absorbent garment of claim 17, wherein the means for extending is attached to at least one of the topsheet and the backsheet.

29. The absorbent garment of claim 17, wherein the means for attaching is attached to the means for extending.

30. The absorbent garment of claim 17, wherein the means for laterally extending comprises a grip attached to the garment inboard of the means for attaching.

31. The absorbent garment of claim 17, wherein a respective portion of the means for extending and a respective portion of a means for attaching comprise opposite ends of a continuous piece of material.

32. The absorbent garment of claim 17, wherein the means for extending further comprises an anchoring means.

33. The absorbent garment of claim 32, wherein the anchoring means comprises a hook and loop fastener.

34. The absorbent garment of claim 17, wherein lateral tensile loads transmitted through the means for extending are distributed substantially evenly throughout the first waist region.

35. A method of making an absorbent garment having a longitudinal dimension and a lateral dimension comprising:
providing a topsheet;
providing a backsheet material to overlay the topsheet and thereby form a first waist region, a second waist region longitudinally opposite the first waist region, and a crotch region therebetween;
disposing an absorbent core between the topsheet and the backsheet;
providing a pair of tabs, each tab extending laterally away from an opposite lateral side of the first waist region;
attaching at least one fastening element to each tab;
attaching one or more target devices to the garment in the second waist region, where the at least one fastening element and the one or more target devices are capable of attaching to one another, the one or more target devices being located so that the first waist region and second waist region of the garment may be joined to one another to secure the garment on an intended wearer;
providing a pair of grips, each grip being attached at an attachment area on laterally opposite sides of the garment in the first waist region, whereby each attachment area is laterally inboard of at least one fastening element attached to each respective tab.

36. The method of claim 35, wherein each tab comprises a portion of at least one of the topsheet and backsheet.

37. The method of claim 35, wherein providing a pair of tabs comprises attaching separate pieces of material to the garment at or near opposite lateral side edges of the first waist region.

38. The method of claim 35, wherein the at least one fastening element comprises a hook portion of a hook and loop fastener and the one or more target devices comprise the loop portion of a hook and loop fastener.

39. The method of claim 35, wherein the at least one fastening element is an adhesive tape and the one or more target devices comprise a tape receiving surface.

40. The method of claim 35, wherein the tabs are relatively wide elasticized tabs.

41. The method of claim 35, wherein the step of providing a pair of grips comprises attaching each grip in an attachment area selected such that the pair of grips may be used to attach the at least one fastening elements to the one or more targeting devices without placing the fastening elements in tension.

42. The method of claim 35, wherein the step of providing a pair of grips comprises attaching each grip in an attachment area comprising one or more attachment points selected to substantially evenly distribute a lateral tensile load on the grips throughout the first waist region.

43. A method of securing a garment to an intended wearer comprising:
placing the crotch region of the garment over the crotch region of the intended wearer such that a first waist region of the garment is adjacent either a front or back waist region of the intended wearer and a second waist region is adjacent an opposing waist region of the intended wearer;
grasping a pair of grips, the grips being located on laterally opposite sides of the first waist region, each grip further being attached to the garment laterally inboard of a respective fastener disposed on the garment;
pulling the grips to extend the laterally opposed sides toward the second waist region in such a manner that the lateral tensile loads on the grips created by the pulling are substantially evenly distributed throughout the first waist region; and
attaching the fasteners to one or more target devices disposed on the garment in the second waist region to thereby secure the garment to the intended wearer.

44. The method of claim 43, wherein pulling the grips further comprises pulling the grips in such a manner that the fasteners are not placed under a lateral tensile load.
